(19) United States
(12) Patent Application Publication
(10) Pub. No.: US 2019/0269350 A1
(43) Pub. Date: Sep. 5, 2019

(54) RESPIRATORY STATE ESTIMATION APPARATUS, RESPIRATORY STATE ESTIMATION METHOD, AND PROGRAM RECORDING MEDIUM

(71) Applicant: Panasonic Intellectual Property Management Co., Ltd., Osaka (JP)

(72) Inventors: YOSHIFUMI HIROSE, Kyoto (JP); SHOICHI ARAKI, Osaka (JP)

(21) Appl. No.: 16/417,737

(22) Filed: May 21, 2019

Related U.S. Application Data
(63) Continuation of application No. PCT/JP2017/018440, filed on May 17, 2017.

(30) Foreign Application Priority Data
Mar. 8, 2017 (JP) 2017-044264

(51) Int. Cl. A61B 5/08; A61B 5/0456; A61B 5/0404; A61B 5/00

(52) U.S. Cl. CPC A61B 5/0826; A61B 5/7253; A61B 5/0404; A61B 5/0456

(57) ABSTRACT

A respiratory state estimation apparatus estimates whether a respiratory state is equivalent to a first respiration including normal respiration or a second respiration smaller in respiratory ventilation volume than the first respiration. The apparatus includes an acquisition unit, a detector, a calculator, and an estimator. The acquisition unit acquires an electrocardiographic waveform of a user. The detector detects amplitudes of R waves in the electrocardiographic waveform. The calculator calculates a spectrum of the amplitudes by performing transform processing with respect to the amplitudes in a time width in which the spectrum has a spectrum shape with a peak in the first respiration and a spectrum shape without a peak in the second respiration. The estimator estimates a respiratory state of the user based on the spectrum.

### TECHNICAL FIELD

The present disclosure relates to a respiratory state estimation apparatus for estimating a respiratory state of a person, a respiratory state estimation method, and a program recording medium.

### BACKGROUND ART

PTL 1 discloses an apneic state determination apparatus that acquires an acoustic signal during sleep and determines an apneic state of a person based on an acquired acoustic signal.

### CITATION LIST

### Patent Literature

PTL 1: Unexamined Japanese Patent Publication No. 2013-202101

### SUMMARY

The present disclosure provides a respiratory state estimation apparatus that can estimate a respiratory state without disturbing respiration.

A respiratory state estimation apparatus according to the present disclosure estimates whether a respiratory state is equivalent to a first respiration including normal respiration or a second respiration smaller in respiratory ventilation volume than the first respiration. The apparatus includes an acquisition unit, a detector, a calculator, and an estimator. The acquisition unit acquires an electrocardiographic waveform of a user. The detector detects amplitudes of R waves in the electrocardiographic waveform. The calculator calculates a spectrum of the amplitudes by performing transform processing with respect to the amplitudes in a time width in which the spectrum has a spectrum shape with a peak in the first respiration and a spectrum shape without a peak in the second respiration. The estimator estimates the respiratory state of the user based on the spectrum.

Note that a general or specific aspect of each of these components may be implemented by a system, a method, an integrated circuit, a computer program, or a recording medium such as a computer-readable CD-ROM or may be implemented by an arbitrary combination of a system, a method, an integrated circuit, a computer program, and a recording medium.

A respiratory state estimation apparatus according to the present disclosure can estimate a respiratory state of a person without disturbing respiration.

### BRIEF DESCRIPTION OF DRAWINGS

FIG. 1 is a schematic view showing an overview of a respiratory state estimation system according to a first exemplary embodiment.

FIG. 2 is a block diagram showing an example of a hardware configuration of a respiratory state estimation apparatus according to the first exemplary embodiment.

FIG. 3 is a block diagram showing an example of a hardware configuration of a wearable device according to the first exemplary embodiment.

FIG. 4 is a block diagram showing an example of a functional configuration of the respiratory state estimation system according to the first exemplary embodiment.

FIG. 5 is a sequence chart showing an example of a respiratory state estimation method in the respiratory state estimation system according to the first exemplary embodiment.

FIG. 6 is a graph showing an example of an electrocardiographic waveform (electrocardiographic waveform information) measured by an electrocardiographic waveform measuring unit.

FIG. 7 is a graph displaying an enlarged electrocardiographic waveform corresponding to two heartbeats extracted from FIG. 6.

FIG. 8 is a graph showing an example of R wave amplitude waveforms detected by a second detector.

FIG. 9 is a graph showing an example of a spectrum in a respiratory component extracted by an extractor.

FIG. 10 is a flowchart showing details of an example of estimation processing.

FIG. 11 is a flowchart showing details of another example of estimation processing.

FIG. 12 is a block diagram showing an example of a hardware configuration of a respiratory state estimation apparatus according to a second exemplary embodiment.

FIG. 13 is a block diagram showing an example of a functional configuration of the respiratory state estimation apparatus according to the second exemplary embodiment.

FIG. 14 is a flowchart showing an example of a respiratory state estimation method in the respiratory state estimation apparatus according to the second exemplary embodiment.

### DESCRIPTION OF EMBODIMENTS

Hereinafter, exemplary embodiments will be described in detail with appropriate reference to the drawings. It is noted that a more detailed description than need may be omitted. For example, the detailed description of already well-known matters and the overlap description of substantially same configurations may be omitted. This is to avoid an unnecessarily redundant description below and to facilitate understanding of a person skilled in the art.

Note that the attached drawings and the following description are provided for those skilled in the art to fully understand the present disclosure, and are not intended to limit the subject matter as described in the appended claims.

### First Exemplary Embodiment

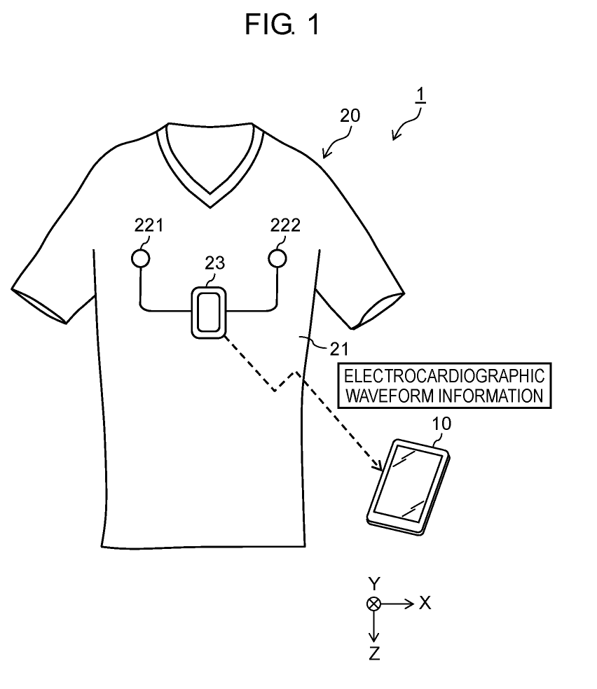

A first exemplary embodiment will be described below with reference to FIGS. 1 to 11.

### [1-1. Configuration]

FIG. 1 is a schematic view showing an overview of a respiratory state estimation system according to the first exemplary embodiment.

More specifically, as shown in FIG. 1, respiratory state estimation system 1 includes respiratory state estimation apparatus 10 and wearable device 20. As shown in FIG. 1, respiratory state estimation apparatus 10 is separated from wearable device 20.

Respiratory state estimation system 1 is a system that estimates a respiratory state of a user by measuring movements in the body (chest) of a user accompanying respiration from an electrocardiographic waveform.

### [1-1-1. Respiratory State Estimation Apparatus]

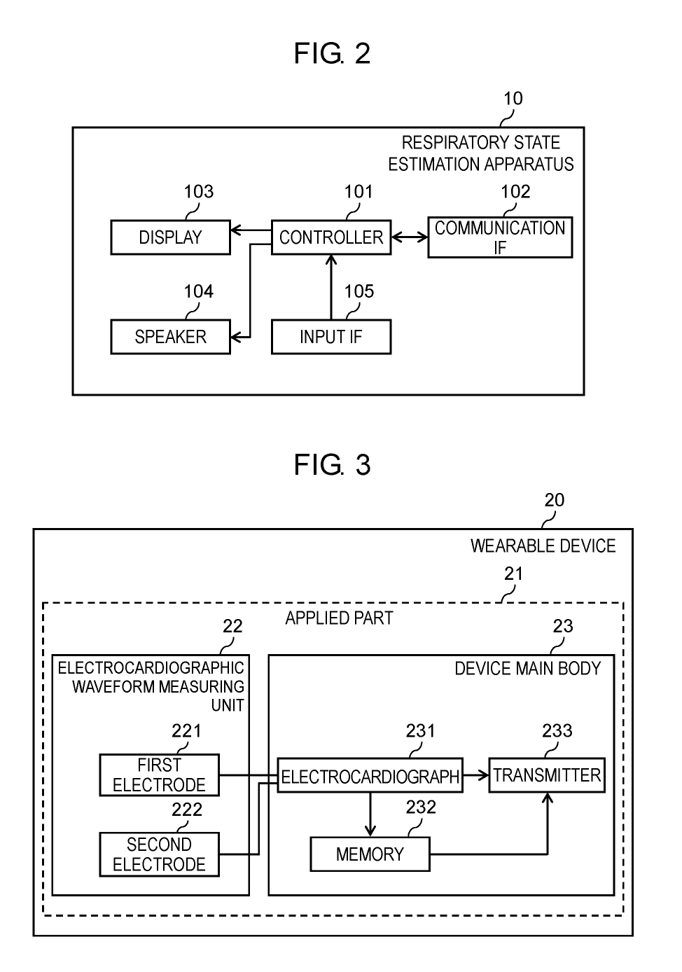

A hardware configuration of respiratory state estimation apparatus 10 will be described with reference to FIG. 2.

FIG. 2 is a block diagram showing an example of the hardware configuration of a respiratory state estimation apparatus according to the first exemplary embodiment.

As shown in FIG. 2, respiratory state estimation apparatus 10 includes controller 101, communication interface (IF) 102, display 103, speaker 104, and input IF 105. Respiratory state estimation apparatus 10 is, for example, a communicable portable device such as a smartphone or tablet device. Note that respiratory state estimation apparatus 10 is a portable device. However, respiratory state estimation apparatus 10 may be an information device such as a personal computer (PC) as long as the apparatus is communicable.

Controller 101 includes a processor that executes control programs for operating respiratory state estimation apparatus 10, a volatile memory area (main memory) used as a work area to be used for the execution of control programs, and a nonvolatile memory area (auxiliary memory) storing control programs, contents, and the like. The volatile memory area is, for example, a random access memory (RAM). The nonvolatile memory area is, for example, a read only memory (ROM), flash memory, or hard disk drive (HDD).

Communication IF 102 is a communication interface that communicates with wearable device 20. Communication IF 102 may be a communication interface corresponding to a transmitter 233 (see FIG. 3) of wearable device 20. That is, communication IF 102 is, for example, a wireless communication interface complying with the Bluetooth (registered trademark) standards. Note that communication IF 102 may be a wireless local area network (LAN) interface complying with the IEEE802.11a/b/g/n standards. In addition, communication IF 102 may be a wireless communication interface complying with communication standards used for a mobile communication system such as third-generation mobile communication system (3G), fourth-generation communication system (4G), or LTE (registered trademark).

Display 103 is a display device that displays processing results obtained by controller 101. Display 103 is, for example, a liquid crystal display or organic EL display.

Speaker 104 is a speaker that outputs sound decoded from audio information.

Input IF 105 is, for example, a touch panel that is arranged on a surface of display 103 and accepts an input from the user to a user interface (UI) displayed on the display 103. Alternatively, input IF 105 may be, for example, an input device such as a ten-key pad or keyboard.

### [1-1-2. Wearable Device]

FIG. 3 is a block diagram showing an example of a hardware configuration of a wearable device according to the first exemplary embodiment.

As shown in FIG. 3, wearable device 20 includes applied part 21, electrocardiographic waveform measuring unit 22, and device main body 23. Applied part 21 is worn on an upper body of the user. Electrocardiographic waveform measuring unit 22 and device main body 23 are arranged on applied part 21.

Applied part 21 is, for example, clothes such as a T-shirt. Applied part 21 is not limited to clothes and may be formed from an extensible belt-like member wound around a chest or an abdominal region of the user.

Electrocardiographic waveform measuring unit 22 includes first electrode 221 and second electrode 222. First electrode 221 and second electrode 222 are electrodes arranged at positions on opposite sides of a heart of the user when viewed from a front side of the user while applied part 21 is worn on the upper body of the user. Note that first electrode 221 and second electrode 222 may not be strictly located at positions on the opposite sides of the heart of the user as long as the electrodes are located near the heart.

Device main body 23 includes electrocardiograph 231, memory 232, and transmitter 233. Device main body 23 is arranged at a predetermined position on applied part 21.

Electrocardiograph 231 is electrically connected to first electrode 221 and second electrode 222 to measure an electrocardiographic waveform of the user. Electrocardiograph 231 outputs electrocardiographic waveform information representing the measured electrocardiographic waveform to transmitter 233.

Transmitter 233 is a communication module that communicates with respiratory state estimation apparatus 10. Transmitter 233 may have, for example, a wireless communication interface complying with the Bluetooth (registered trademark) standards or a wireless local area network (LAN) interface complying with the IEEE802.11a/b/g/n standards.

Memory 232 stores electrocardiographic waveform information representing the electrocardiographic waveform measured by electrocardiograph 231. When transmitter 233 is communicably connected to respiratory state estimation apparatus 10, transmitter 233 may read out electrocardiographic waveform information stored in memory 232 and transmit the readout electrocardiographic waveform information to respiratory state estimation apparatus 10.

### [1-2. Functional Configuration of Respiratory State Estimation System]

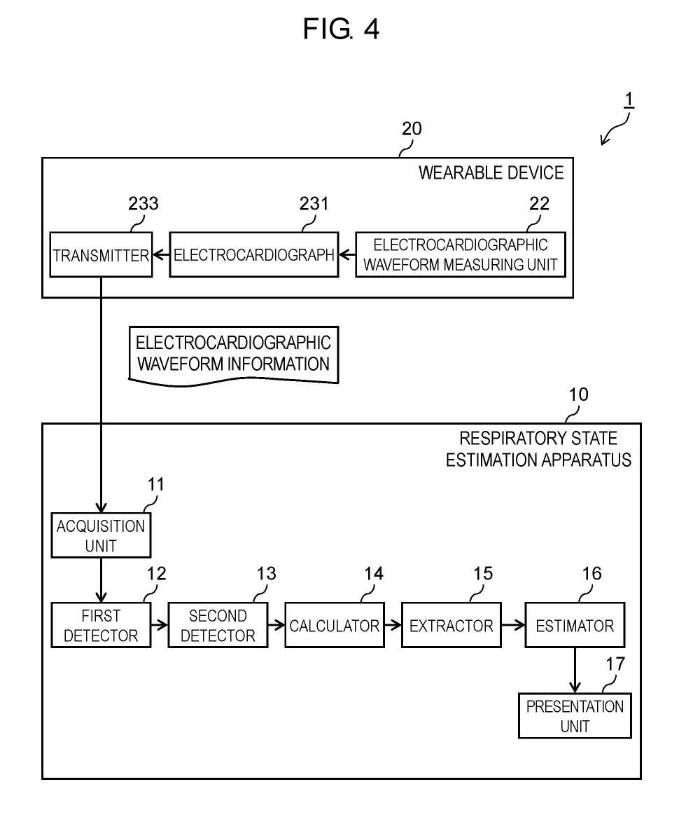

The functional configuration of respiratory state estimation system 1 will be described next with reference to FIG. 4.

FIG. 4 is a block diagram showing an example of the functional configuration of the respiratory state estimation system according to the first exemplary embodiment.

A functional configuration of wearable device 20 will be described first.

Wearable device 20 includes electrocardiographic waveform measuring unit 22, electrocardiograph 231, and transmitter 233, which constitute the functional configuration.

Electrocardiographic waveform measuring unit 22 measures an electrocardiographic waveform of the user. Electrocardiographic waveform measuring unit 22 measures the electrocardiographic waveform of the user and generates electrocardiographic waveform information representing the electrocardiographic waveform. Electrocardiographic waveform measurement is implemented by, for example, electrocardiographic waveform measuring unit 22, a plurality of electrodes 221, 222, and electrocardiograph 231.

Transmitter 233 transmits the generated electrocardiographic waveform information to respiratory state estimation apparatus 10. Note that transmitter 233 transmits electrocardiographic waveform information stored in memory 232 to respiratory state estimation apparatus 10 in a predetermined cycle. Transmitter 233 is implemented by, for example, a communication module. That is, transmitter 233 transmits electrocardiographic waveform information to respiratory state estimation apparatus 10 to which memory 232 is communicably connected via, for example, Bluetooth (registered trademark).

A functional configuration of respiratory state estimation apparatus 10 will be described next.

Respiratory state estimation apparatus 10 includes acquisition unit 11, first detector 12, second detector 13, calculator 14, extractor 15, estimator 16, and presentation unit 17.

Acquisition unit 11 receives electrocardiographic waveform information received from transmitter 233 of wearable device 20. That is, acquisition unit 11 communicates with wearable device 20 worn on the body of the user while having electrocardiograph 231. With this operation, acquisition unit 11 acquires electrocardiographic waveform information representing an electrocardiographic waveform of the user. Acquisition unit 11 is implemented by, for example, controller 101 and communication IF 102.

First detector 12 detects R waves in the electrocardiographic waveform represented by the electrocardiographic waveform information acquired by acquisition unit 11. More specifically, first detector 12 detects a plurality of R waves appearing at different times in the electrocardiographic waveform represented by the electrocardiographic waveform information. First detector 12 is implemented by, for example, controller 101.

Second detector 13 detects amplitudes of R waves detected by first detector 12. More specifically, by detecting amplitudes (peaks) of a plurality of R waves detected by first detector 12 and times when the amplitudes appear, second detector 13 detects the amplitudes of the R waves associated with the times. Second detector 13 outputs, to calculator 14, amplitude information representing the detected amplitudes of the plurality of R waves respectively associated with the times. In addition, second detector 13 generates an R wave amplitude waveform representing changes in amplitudes of R waves by using the plurality of amplitudes of the R waves associated with the times. Second detector 13 re-samples amplitudes of R waves in a predetermined sampling cycle by using the R wave amplitude waveform. This enables second detector 13 to obtain a plurality of amplitudes of R waves in a predetermined sampling cycle. Second detector 13 is implemented by, for example, controller 101.

Calculator 14 calculates a spectrum of the amplitudes of the R waves detected by second detector 13. Calculator 14 performs transform processing of transforming the amplitudes of the plurality of R waves obtained by re-sampling into frequency spectrum information. Calculator 14 transforms the amplitudes of the R waves into spectrum information of a frequency domain of the amplitudes of the R waves by performing fast Fourier transform (FFT).

Calculator 14 may execute FFT processing, for example, in a time width (about 2 seconds to 20 seconds) corresponding to one respiration cycle to 10 respiration cycles. Note that this time width indicates each cycle when FFT processing is repeatedly executed. In this case, shortening the time width in which FFT processing is executed will increase a following property of spectrum information with respect to a change in respiratory rate but decrease resistance of the spectrum information against noise such as body motion (spectrum information responds sensitively to noise). In contrast, increasing the time width will increase the resistance of the spectrum information against noise such as body motion but decrease the following property of the spectrum information with respect to a change in respiratory rate. Accordingly, it is preferable to properly adjust and determine a time width in which FFT processing is to be executed. In addition, it is preferable to use a window function such as Hanning window when executing FFT processing.

Calculator 14 is implemented by, for example, controller 101.

Extractor 15 extracts a respiratory component in a predetermined frequency band from a spectrum calculated by calculator 14. Extractor 15 extracts a respiratory component by extracting a preset frequency component from the calculated spectrum. Assuming that a respiratory rate is 5/min to 30/min, extractor 15 extracts a spectrum in a frequency band between 0.08 Hz and 0.5 Hz (inclusive) as a respiratory component. In this manner, extractor 15 extracts a respiratory component in a frequency band determined based on respiration of a user. This enables estimator 16 to prevent erroneous estimation when noise mixes in a portion outside the frequency band in next estimation processing.

Extractor 15 is implemented by, for example, controller 101.

Estimator 16 estimates a respiratory state of a user from a respiratory component extracted by extractor 15. That is, estimator 16 estimates the respiratory state of the user by setting the respiratory component extracted by extractor 15 as an index value. More specifically, when a peak intensity of a spectrum of a respiratory component extracted by extractor 15 is more than or equal to a predetermined intensity, estimator 16 may estimate that a respiratory state is equivalent to deep respiration. In addition, when the peak intensity of the spectrum of the respiratory component extracted by extractor 15 is less than the predetermined intensity, estimator 16 may estimate that a respiratory state is equivalent to hypopnea or apnea. Furthermore, when a standard deviation of a spectrum of a respiratory component is more than or equal to a predetermined standard deviation, estimator 16 may estimate that a respiratory state is equivalent to deep respiration. Moreover, when the standard deviation of the spectrum of the respiratory component is less than the predetermined standard deviation, estimator 16 may estimate that a respiratory state is equivalent to hypopnea or apnea.

Note that a spectrum intensity of an R wave amplitude accompanying respiratory motion differs depending on conditions such as positions of electrodes 221, 222, and hence is preferably set as appropriate.

Note that hypopnea indicates a state in which the respiratory ventilation volume is low. Hypopnea in medical terms indicates that a respiratory gas flow or respiratory motion decreases to less than 70% of a predetermined reference and a respiratory event accompanying a reduction in oxygen saturation of 4% or more continues for 10 seconds or more. In the present disclosure, as an index for detecting such a state, a peak intensity of a spectrum of a respiration band or a standard deviation of a spectrum is used. Deep respiration is equivalent to a state in which the above respiratory gas flow or respiratory motion satisfies the predetermined reference.

Estimator 16 is implemented by, for example, controller 101.

Presentation unit 17 displays an image or character information representing a respiratory state estimated by estimator 16. Presentation unit 17 may output a sound representing the estimated respiratory state. Presentation unit 17 may be implemented by, for example, controller 101 and display 103 or may be implemented by controller 101 and speaker 104.

### [1-2. Operation]

An operation of respiratory state estimation system 1 having the above configuration will be described below. That is, a respiratory state estimation method performed by respiratory state estimation system 1 will be described.

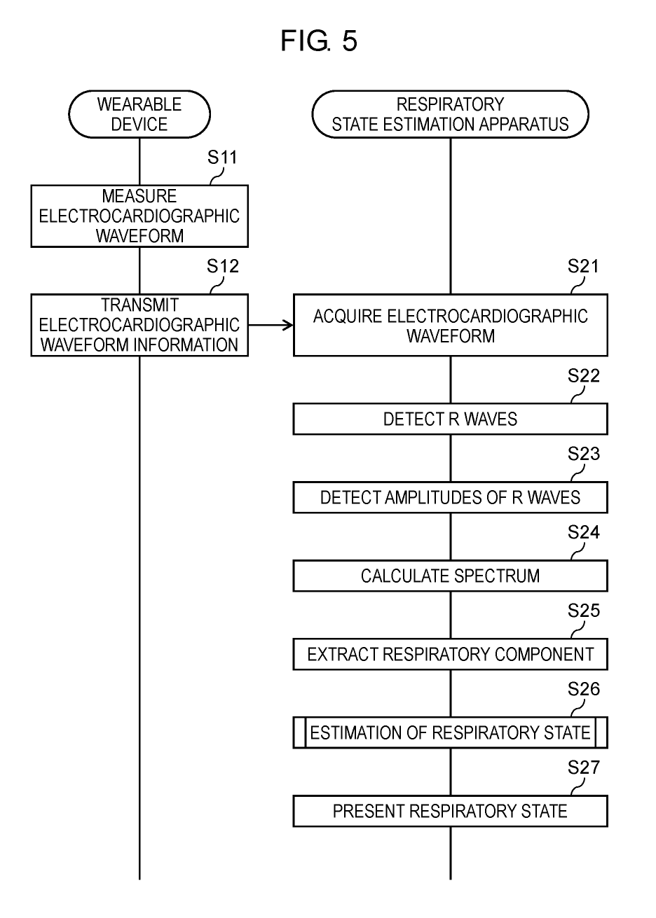

FIG. 5 is a sequence chart showing an example of the respiratory state estimation method in respiratory state estimation system 1 according to the first exemplary embodiment.

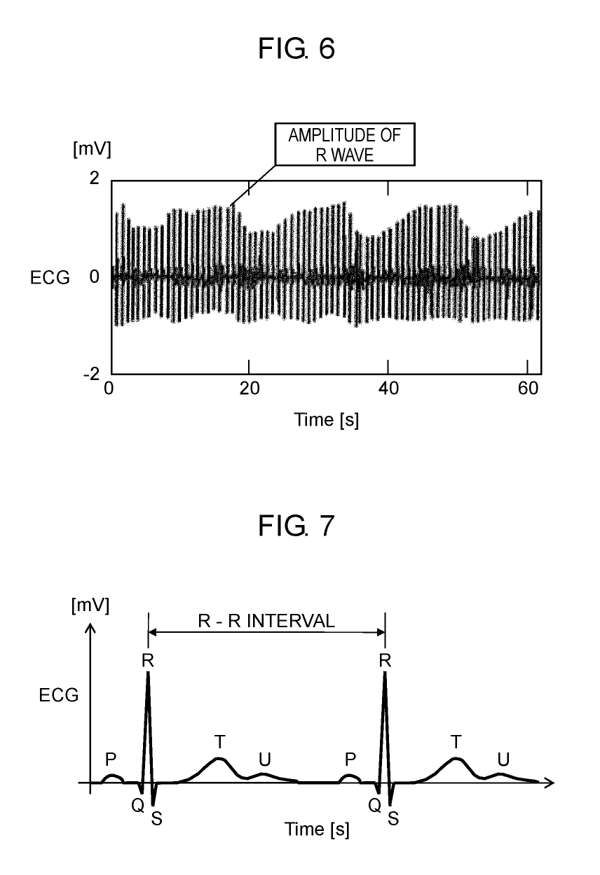

In wearable device 20 worn on the body of the user, electrocardiographic waveform measuring unit 22 measures an electrocardiographic waveform of the user (S11). With this operation, electrocardiographic waveform measuring unit 22 acquires, for example, an electrocardiographic waveform as that shown in FIG. 6.

FIG. 6 is a graph showing an example of an electrocardiographic waveform (electrocardiographic waveform information) measured by the electrocardiographic waveform measuring unit. FIG. 7 is a graph displaying an enlarged electrocardiographic waveform corresponding to two heartbeats extracted from FIG. 6. Referring to FIGS. 6 and 7, an abscissa represents time [s], and an ordinate represents electrocardiogram (ECG) [mV]. In general, in an electrocardiographic waveform, a P wave, Q wave, R wave, S wave, T wave, and U wave appear for each heartbeat. Of these waves, an R wave has a large amplitude and exhibits a steep change per unit, and hence is used for heartbeat detection. Referring to FIG. 7, a portion indicated by “R” is an R wave, and an R-R interval including two R waves corresponds to one heartbeat time.

In wearable device 20, transmitter 233 then transmits electrocardiographic waveform information to respiratory state estimation apparatus 10 (S12).

In respiratory state estimation apparatus 10, acquisition unit 11 receives the electrocardiographic waveform information transmitted from transmitter 233 of wearable device 20. With this operation, acquisition unit acquires an electrocardiographic waveform represented by the electrocardiographic waveform information (S21).

First detector 12 then detects R waves of the electrocardiographic waveform acquired by acquisition unit 11 (S22).

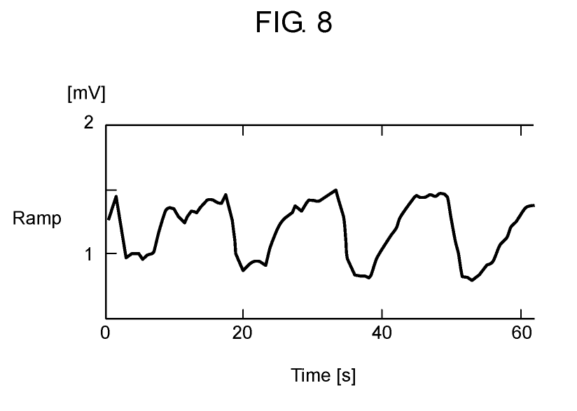

Second detector 13 detects amplitudes of the R waves detected by first detector 12 (S23). More specifically, second detector 13 generates an R wave amplitude curve representing temporal changes in R wave amplitude. With this operation, second detector 13 generates an R wave amplitude curve as that shown in FIG. 8.

FIG. 8 is a graph showing an example of an R wave amplitude waveform detected by second detector 13. Referring to FIG. 8, the abscissa represents time [s], and the ordinate represents R wave amplitude [mV].

As shown in FIG. 8, when the user breathes, a chest of the user moves, and a capacity of a lung changes, resulting in a change in impedance between the plurality of electrodes 221, 222. Accordingly, even R waves from the same user differ in amplitude in accordance with a displacement of the chest of the user accompanying respiration. That is, generating a waveform representing temporal changes in R waves can estimate motion of the chest caused by respiration of the user.

Second detector 13 detects a plurality of R wave amplitudes in a predetermined sampling cycle by re-sampling R wave amplitudes in the predetermined sampling cycle using an R wave amplitude waveform.

Calculator 14 then calculates a spectrum of the R wave amplitudes detected by second detector 13 (S24).

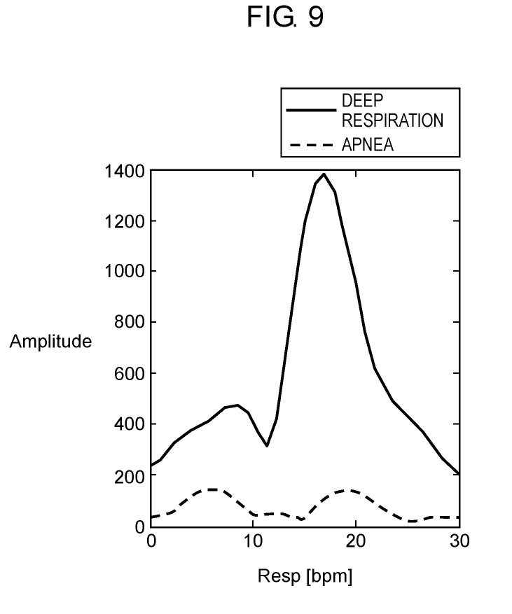

Subsequently, extractor 15 extracts a respiratory component in a frequency band of respiration of the user from the spectrum calculated by calculator 14 (S25). More specifically, extractor 15 extracts, as a respiratory component, a spectrum of a predetermined frequency band (for example, between 0.08 Hz and 0.5 Hz (inclusive)) of the spectrum calculated by calculator 14. With this operation, extractor 15 extracts, for example, a spectrum as that shown in FIG. 9 in a respiratory component.

FIG. 9 is a graph showing an example of a spectrum in a respiratory component which is extracted by extractor 15. Referring to FIG. 9, the abscissa represents frequency, and the ordinate represents intensity.

As shown in FIG. 9, when the user takes a deep respiration including a normal respiration, a peak appears between 5 bpm and 30 bpm. A peak intensity of the peak exceeds a predetermined peak intensity (for example, 800). In contrast, when the user takes apnea (or hypopnea), a spectrum is flat, and no noticeable peak appears.

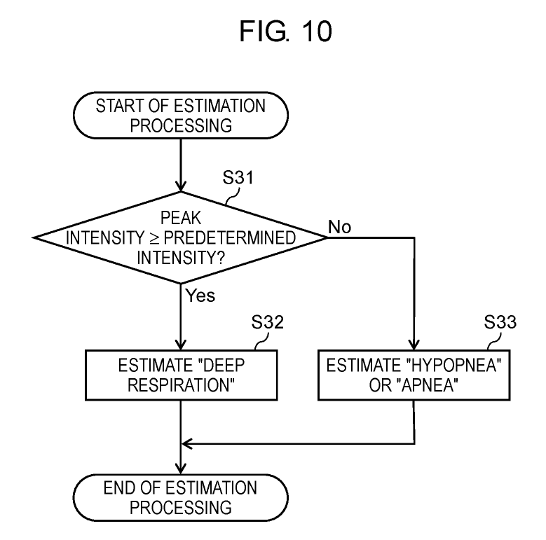

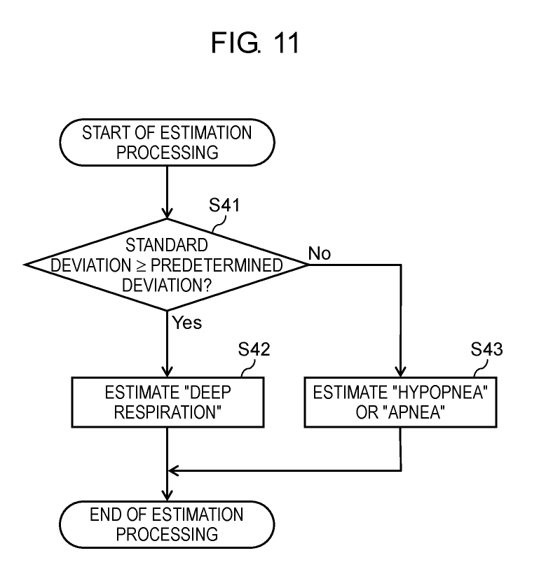

Subsequently, estimator 16 estimates a respiratory state of the user from a respiratory component extracted by extractor 15 (S26). Details of respiratory state estimation processing by estimator 16 will be described with reference to FIGS. 10 and 11.

FIG. 10 is a flowchart showing details of an example of estimation processing.

Upon completion of step S25 described above, estimator 16 determines whether a peak intensity of a spectrum calculated by calculator 14 is more than or equal to a predetermined intensity (S31).

If the peak intensity of the spectrum is more than or equal to the predetermined intensity (Yes in S31), estimator 16 estimates that a respiratory state of the user is equivalent to deep respiration (S32).

In contrast, upon determining that the peak intensity of the spectrum is less than the predetermined intensity (No in S31), the estimator 16 estimates that the respiratory state of the user is equivalent to hypopnea or apnea (S33).

As shown in FIG. 9, there is a noticeable difference in peak spectrum intensity between deep respiration and apnea (or hypopnea). Accordingly, comparing the peak spectrum intensity with the predetermined peak intensity can determine whether the respiratory state from which the spectrum is obtained is equivalent to deep respiration or apnea (or hypopnea).

Estimator 16 may perform estimation processing shown in a flowchart of FIG. 11 instead of a flowchart of FIG. 10.

FIG. 11 is a flowchart showing details of another example of estimation processing.

Upon completion of step S25 described above, estimator 16 determines whether a standard deviation of a spectrum calculated by calculator 14 is more than or equal to a predetermined standard deviation (S41).

Upon determining that the standard deviation of the spectrum is more than or equal to the predetermined standard deviation (Yes in S41), estimator 16 estimates that the respiratory state of the user is equivalent to deep respiration (S42).

In contrast, upon determining that the standard deviation of the spectrum is less than the predetermined standard deviation (No in S41), estimator 16 estimates that the respiratory state of the user is equivalent to hypopnea or apnea (S43).

As shown in FIG. 9, spectrum peaks appear differently between deep respiration and apnea (or hypopnea). That is, a spectrum peak occurs in deep respiration, whereas a spectrum becomes flat and no peak occurs in apnea (or hypopnea). Accordingly, comparing a standard deviation of a spectrum with a predetermined standard deviation makes it possible to determine whether a respiratory state from which the spectrum is obtained is equivalent to apnea (or hypopnea).

Note that estimator 16 may estimate a respiratory state of a user by using both the flowchart of FIG. 10 and the flowchart of FIG. 11. In this case, if both results obtained by the flowchart of FIG. 10 and the flowchart of FIG. 11 indicate deep respiration, estimator 16 may determine that the respiratory state of the user is equivalent to deep respiration. If either of results obtained by the flowchart of FIG. 10 and the flowchart of FIG. 11 indicates apnea (or hypopnea), estimator 16 may determine that the respiratory state of the user is equivalent to apnea (or hypopnea).

Presentation unit 17 presents information (an image, text, or sound) representing a respiratory state estimated by estimator 16 (S27).

### [1-3. Effects and the Like]

As described above, in this exemplary embodiment, respiratory state estimation apparatus 10 includes acquisition unit 11, first detector 12, second detector 13, calculator 14, extractor 15, and estimator 16. Acquisition unit 11 acquires an electrocardiographic waveform of the user. First detector 12 and second detector 13 detect amplitudes of R waves in the electrocardiographic waveform acquired by acquisition unit 11. Calculator 14 calculates a spectrum of the amplitude detected by first detector 12 and second detector 13. Extractor 15 extracts a respiratory component in a predetermined frequency band from a spectrum calculated by calculator 14. Estimator 16 estimates a respiratory state of a user from a respiratory component extracted by extractor 15. In addition, estimator 16 estimates a respiratory state of the user by using a respiratory component extracted by extractor 15 as an index value.

This makes it possible to estimate a respiratory state of the user without disturbing respiration.

In this exemplary embodiment, if a peak intensity of a spectrum of a respiratory component extracted by extractor 15 is more than or equal to a predetermined intensity, estimator 16 estimates that the respiratory state is equivalent to deep respiration. If the peak intensity of the spectrum of the respiratory component extracted by extractor 15 is less than the predetermined intensity, estimator 16 estimates that the respiratory state is equivalent to hypopnea or apnea.

This makes it possible to effectively estimate the respiratory state of the user.

In this exemplary embodiment, if a standard deviation of a spectrum of a respiratory component is more than or equal to a predetermined standard deviation, estimator 16 determines that a respiratory state is equivalent to deep respiration. In addition, if the standard deviation of the spectrum of the respiratory component is less than the predetermined standard deviation, estimator 16 determines that a respiratory state is equivalent to apnea or hypopnea.

This makes it possible to effectively estimate the respiratory state of the user.

### [1-4. First Modification]

The first exemplary embodiment is configured such that device main body 23 is separate from first electrode 221 and second electrode 222 arranged on applied part 21, and is electrically connected to first electrode 221 and second electrode 222. However, this is not exhaustive. For example, device main body 23 may have first electrode 221 and second electrode 222 so as to be integrated with first electrode 221 and second electrode 222. In this case, device main body 23 integrated with first electrode 221 and second electrode 222 may be fixed to clothes serving as applied part 21 of the user so as to function as wearable device 20.

### [1-5. Second Modification]

According to the first exemplary embodiment described above, estimator 16 estimates a respiratory state of a user based on whether a peak intensity of a spectrum of a respiratory component extracted by extractor 15 is more than or equal to a predetermined intensity or whether a standard deviation of the spectrum is more than or equal to a predetermined standard deviation. However, this is not exhaustive. Estimator 16 may estimate the respiratory state of the user by comparing a second respiratory component of the user, obtained by processing based on an electrocardiographic waveform measured in a time width (about 2 seconds to 20 seconds) corresponding to one respiration cycle to 10 respiration cycles with a first respiratory component of the user, obtained by processing based on an electrocardiographic waveform measured over a predetermined time (for example, 1 hour) or more before the first respiratory component.

A second respiratory component is data measured in real time in a period shorter than that of a first respiratory component. For this reason, if the second respiratory component includes a hypopnea or apnea state, a value of the second respiratory state noticeably differs from a value of the first respiratory component. Accordingly, if a second intensity as a peak intensity of a spectrum of the second respiratory component is more than or equal to a first peak intensity as a peak intensity of a spectrum of a first respiratory component, estimator 16 may determine that a respiratory state of the user is a deep respiratory state. In addition, if the second peak intensity is less than the first peak intensity, estimator 16 may determine that the respiratory state of the user is equivalent to hypopnea or apnea. Furthermore, if a second standard deviation as a standard deviation of a spectrum of the second respiratory component is more than or equal to a first standard deviation as a standard deviation of a spectrum of the first respiratory component, estimator 16 determines that the respiratory state of the user is a deep respiratory state. Moreover, if the second standard deviation is less than the first standard deviation, estimator 16 may determine that the respiratory state of the user is equivalent to hypopnea or apnea.

In this manner, estimator 16 may estimate the respiratory state of the user in the second period shorter than the first period by comparing the first respiratory component of the user, output by the processing by first detector 12, second detector 13, calculator 14, and extractor 15 based on the electrocardiographic waveform measured over the first period, with the second respiratory component of the user, output by the processing by first detector 12, second detector 13, calculator 14, and extractor 15 based on the electrocardiographic waveform measured over the second period.

This makes it possible to determine a respiratory state based on an electrocardiographic waveform acquired from the same user, thereby performing determination in accordance with characteristics of the user. In addition, because the first period is longer than the second period, the first respiratory component is averaged more than the second respiratory component. This enables estimator 16 to estimate the respiratory state of the user in the second period by comparing the first respiratory component with the second respiratory component.

### [1-6. Third Modification]

Note that in this exemplary embodiment, respiratory state estimation apparatus 10 includes first detector 12 that detects R waves in an electrocardiographic waveform acquired by acquisition unit 11 and second detector 13 that detects amplitudes of the R waves detected by first detector 12. However, the present disclosure is not limited to this. Respiratory state estimation apparatus 10 may include only one detector, which may detect amplitudes of R waves in an electrocardiographic waveform acquired by acquisition unit 11.

### [1-7. Fourth Modification]

Note that in this exemplary embodiment, first electrode 221 and second electrode 222 are arranged on a front surface of the upper body of the user. However, the present disclosure is not limited to this. First electrode 221 may be arranged on the front surface of the upper body of the user and second electrode 222 may be arranged on a rear surface of the upper body of the user such that a plurality of electrodes 221, 222 are arranged at positions on opposite sides of the heart of the user. That is, arranging the plurality of electrodes 221, 222 at positions on opposite sides of the heart of the user means arranging the plurality of electrodes 221, 222 so as to cause a current flowing between the plurality of electrodes 221, 222 to pass through the heart of the user.

### [1-8. Fifth Modification]

Note that in this exemplary embodiment, acquisition unit 11 acquires an electrocardiographic waveform from wearable device 20. However, the present disclosure is not limited to this. Acquisition unit 11 may acquire an electrocardiographic waveform from a recording medium recording an electrocardiographic waveform of a user.

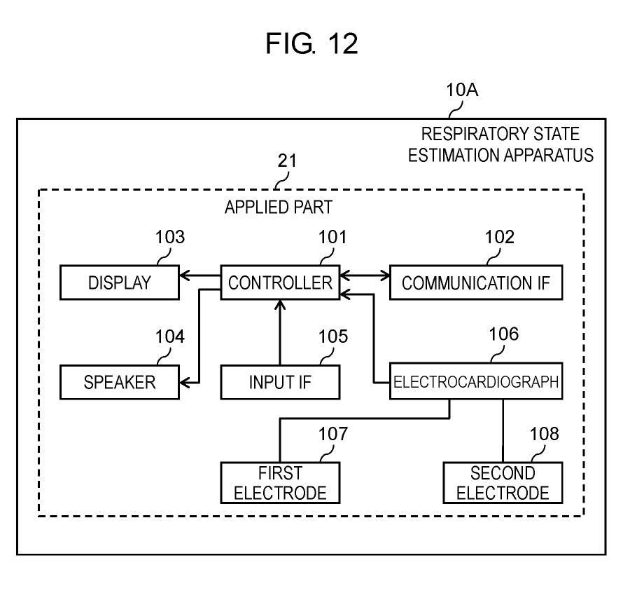

### Second Exemplary Embodiment The second exemplary embodiment will be described below with reference to FIGS. 12 to 14.

### [2-1. Configuration]

FIG. 12 is a block diagram showing an example of a hardware configuration of a respiratory state estimation apparatus according to the second exemplary embodiment.

As shown in FIG. 12, unlike in the first exemplary embodiment, in the second exemplary embodiment, respiratory state estimation apparatus 10A performs all processing in a respiratory state estimation method. That is, respiratory state estimation apparatus 10A according to the second exemplary embodiment additionally includes electrocardiograph 106, first electrode 107, and second electrode 108 as compared with respiratory state estimation apparatus 10 according to the first exemplary embodiment. Electrocardiograph 106, first electrode 107, and second electrode 108 respectively have the same configurations as those of electrocardiograph 231, first electrode 221, and second electrode 222. Other components are the same as those of the first exemplary embodiment, and hence will be denoted by the same reference numerals as those in the first exemplary embodiment. A description of these components will be omitted.

In this case, respiratory state estimation apparatus 10A may not include display 103 and communication IF 102. In addition, respiratory state estimation apparatus 10A may be implemented as a wearable device including applied part 21 as shown in FIG. 12.

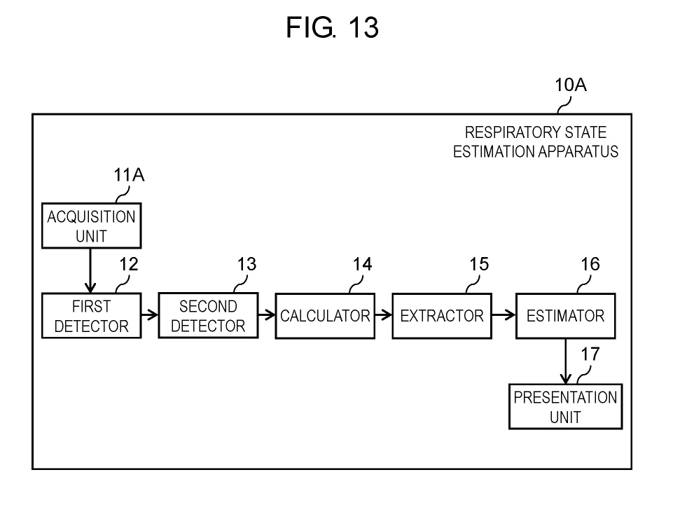

FIG. 13 is a block diagram showing an example of a functional configuration of the respiratory state estimation apparatus according to the second exemplary embodiment.

As shown in FIG. 13, unlike in the first exemplary embodiment, in the second exemplary embodiment, acquisition unit 11A is implemented by electrocardiograph 106, first electrode 107, and second electrode 108. That is, acquisition unit 11A acquires an electrocardiographic waveform of a user by measuring the electrocardiographic waveform of the user.

Components other than acquisition unit 11A are the same as those of the first exemplary embodiment, and hence will be denoted by the same reference numerals as those in the first exemplary embodiment. A description of these components will be omitted.

### [2-2. Operation]

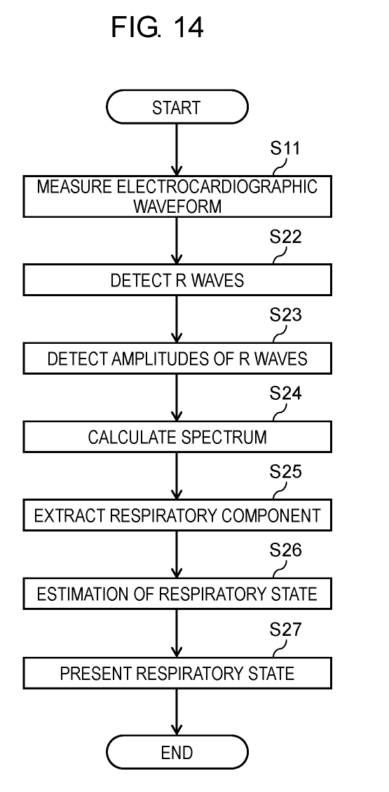

FIG. 14 is a flowchart showing an example of a respiratory state estimation method in the respiratory state estimation apparatus according to the second exemplary embodiment.

As shown in FIG. 14, an operation of respiratory state estimation apparatus 10A according to the second exemplary embodiment differs from an operation of respiratory state estimation system 1 according to the first exemplary embodiment that all processing is completed within respiratory state estimation apparatus 10A. That is, in a sequence chart described with reference to FIG. 5, steps S12 and S21 are omitted.

That is, respiratory state estimation apparatus 10A performs step S22 after performing step S11. Accordingly, respiratory state estimation apparatus 10A performs processing associated with measurement of an electrocardiographic waveform, detection of R waves, detection of amplitudes of R waves, calculation of a spectrum, extraction of a respiratory component, and estimation of a respiratory state.

### [2-3. Effects]

As described above, in this exemplary embodiment, respiratory state estimation apparatus 10A further includes a plurality of electrodes 107 and 108 that are attached to a chest of a user. Acquisition unit 11A acquires an electrocardiographic waveform of the user from the plurality of electrodes 107 and 108 attached to the chest of the use0

This makes it possible to accurately acquire the electrocardiographic waveform of the use

0 In this exemplary embodiment, respiratory state estimation apparatus 10A further includes applied part 21 that is attached to the upper body of the user. Applied part 21 has the plurality of electrodes 107 and 108 arranged at positions on opposite sides of the heart of the user while being attached to the upper body of the use0

Accordingly, only attaching applied part 21 to the upper body of the user can arrange the plurality of electrodes 107 and 108 at proper positions on the chest of the use

0 Although the plurality of electrodes 107 and 108 are attached to the chest of the user, the present disclosure is not limited to this. The plurality of electrodes 107 and 108 may be attached to a portion of the upper body of the user other than the chest of the user. For example, the plurality of electrodes 107 and 108 may be attached to an arm or hand of the user.

Note that transform processing to a frequency domain is not limited to FFT processing and may be discrete Fourier transform (DFT) processing, discrete cosine transform (DCT) processing, wavelet transform processing.

An estimation result on a respiratory state of the user which has been estimated in the above manner may be transmitted to a server (not shown) via a network. Alternatively, such information may be accumulated in a memory (not shown).

Note that in each exemplary embodiment described above, each constituent element may be implemented by dedicated hardware or by executing a software program suitable for each constituent element. Each constituent element may be implemented by causing a program executor such as a central processing unit (CPU) or processor to read out and execute a software program recorded on a recording medium such as a hard disk or semiconductor memory. In this case, software that implements the respiratory state estimation apparatus according to each exemplary embodiment described above includes the following programs.

That is, this program causes a computer to execute a respiratory state estimation method including acquiring an electrocardiographic waveform of a user, detecting amplitudes of R waves in the electrocardiographic waveform acquired in the acquiring, calculating a spectrum of the amplitudes detected in the detecting, extracting a respiratory component in a predetermined frequency band from the spectrum calculated in the calculating, and estimating a respiratory state of the user from the respiratory component extracted in the extracting.

Although the respiratory state estimation apparatuses and the like according to one or a plurality of aspects of the present disclosure have been described based on the exemplary embodiments, the present disclosure is not limited to the exemplary embodiments. The present disclosure may incorporate, in one or a plurality of aspects of the present disclosure, exemplary embodiments obtained by applying various modifications conceived by persons skilled in the art and exemplary embodiments obtained by combining constituent elements in different exemplary embodiments.

As described above, each exemplary embodiment has been described as an example of a technique according to the present disclosure. The attached drawings and detailed descriptions have been provided for this purpose.

Accordingly, the constituent elements described in the attached drawings and detailed descriptions may include not only constituent elements that are essential to solve the problem but also constituent elements that are provided as examples used to exemplify the technique and are not essential to solve the problem. For this reason, the fact that the constituent elements that are not essential are described in the attached drawings and detailed descriptions should not directly be interpreted to indicate that the inessential constituent elements are essential.

Each exemplary embodiment described above is provided to exemplify the technique according to the present disclosure. Therefore, it is possible to make various changes, replacements, additions, omissions, and the like within the scope of the claims and equivalents thereof.

### INDUSTRIAL APPLICABILITY

The present disclosure can be applied to a respiratory state estimation apparatus that can estimate a respiratory state of a person without disturbing respiration.

### REFERENCE MARKS IN THE DRAWINGS

1: respiratory state estimation system

10, 10A: respiratory state estimation apparatus

11, 11A: acquisition unit

12: first detector

13: second detector

14: calculator

15: extractor

16: estimator

17: presentation unit

20: wearable device

22: electrocardiographic waveform measuring unit

23: device main body

101: controller

102: communication IF

103: display

104: speaker

105: input IF

106, 231: electrocardiograph

107, 221: first electrode

108, 222: second electrode

21: applied part

232: memory

233: transmitter

1. A respiratory state estimation apparatus that estimates whether a respiratory state is equivalent to a first respiration including normal respiration or a second respiration smaller in respiratory ventilation volume than the first respiration, the respiratory state estimation apparatus comprising:
an acquisition unit configured to acquire an electrocardiographic waveform of a user;
a detector configured to detect amplitudes of R waves in the electrocardiographic waveform;
a calculator configured to calculate a spectrum of the amplitudes by performing transform processing with respect to the amplitudes in a time width in which the spectrum has a spectrum shape with a peak in the first respiration and a spectrum shape without a peak in the second respiration; and
an estimator configured to estimate a respiratory state of the user based on the spectrum.

2. The respiratory state estimation apparatus according to claim 1, wherein the time width is between 2 seconds and 20 seconds, inclusive.

3. The respiratory state estimation apparatus according to claim 1, further comprising an extractor configured to extract a first frequency band from the spectrum,
wherein the estimator estimates the respiratory state based on the spectrum of the first frequency band.

4. The respiratory state estimation apparatus according to claim 3, wherein the first frequency band is a frequency band corresponding to the first respiration.

5. The respiratory state estimation apparatus according to claim 3, wherein the first frequency band falls within a range of not more than 0.5 Hz.

6. The respiratory state estimation apparatus according to claim 3, wherein the first frequency band falls within a range of not less than 0.08 Hz.

7. The respiratory state estimation apparatus according to claim 1, wherein
the estimator estimates that the respiratory state is equivalent to the first respiration when a peak intensity of the spectrum is not less than a predetermined value, and
estimates that the respiratory state is equivalent to the second respiration when the peak intensity is less than the predetermined value.

8. The respiratory state estimation apparatus according to claim 1, wherein
the estimator estimates that the respiratory state is equivalent to the first respiration when a standard deviation of the spectrum is not less than a predetermined standard deviation, and
estimates that the respiratory state is equivalent to the second respiration when the standard deviation is less than the predetermined standard deviation.

9. The respiratory state estimation apparatus according to claim 8, wherein the estimator estimates that the respiratory state is equivalent to the second respiration instead of the first respiration when the standard deviation is not less than the predetermined standard deviation and a peak intensity of a spectrum in the first frequency band is less than a predetermined intensity.

10. The respiratory state estimation apparatus according to claim 1, wherein the estimator estimates the respiratory state in a second period shorter than a first period by comparing a first spectrum of the user with a second spectrum of the user, the first spectrum being calculated by the calculator based on the electrocardiographic waveform measured over the first period, the second spectrum being calculated by the calculator based on the electrocardiographic waveform measured over the second period.

11. The respiratory state estimation apparatus according to claim 10, wherein
the estimator estimates that the respiratory state is equivalent to deep respiration when a second peak intensity of the second spectrum is not less than a first peak intensity of the first spectrum, and
estimates that the respiratory state is equivalent to hypopnea or apnea when the second peak intensity is less than the first peak intensity.

12. The respiratory state estimation apparatus according to claim 10, wherein
the estimator estimates that the respiratory state is equivalent to deep respiration when a second standard deviation of the second spectrum is not less than a first standard deviation of the first spectrum, and
estimates that the respiratory state is equivalent to hypopnea or apnea when the second standard deviation is less than the first standard deviation.

13. The respiratory state estimation apparatus according to claim 1, wherein the acquisition unit acquires the electrocardiographic waveform of the user from a recording medium recording the electrocardiographic waveform.

14. The respiratory state estimation apparatus according to claim 1, further comprising a plurality of electrodes that are attached to an upper body of the user,
wherein the acquisition unit acquires the electrocardiographic waveform of the user from the plurality of electrodes.

15. The respiratory state estimation apparatus according to claim 14, further comprising an applied part that is attached to the upper body of the user, the applied part having the plurality of electrodes arranged at positions on opposite sides of a heart of the user while the applied part is attached to the upper body of the user.

16. A respiratory state estimation method of estimating whether a respiratory state is equivalent to a first respiration including normal respiration or a second respiration smaller in respiratory ventilation volume than the first respiration, the respiratory state estimation method comprising:
acquiring an electrocardiographic waveform of a user;
detecting amplitudes of R waves in the electrocardiographic waveform;
calculating a spectrum of the amplitudes by performing transform processing with respect to the amplitudes in a time width in which the spectrum has a spectrum shape with a peak in the first respiration and a spectrum shape without a peak in the second respiration; and
estimating a respiratory state of the user based on the spectrum.

17. A program recording medium recording a program for causing a computer to execute a respiratory state estimation method of estimating whether a respiratory state is equivalent to a first respiration including normal respiration or a second respiration smaller in respiratory ventilation volume than the first respiration, the respiratory state estimation method including
acquiring an electrocardiographic waveform of a user,
detecting amplitudes of R waves in the electrocardiographic waveform,
calculating a spectrum of the amplitudes by performing transform processing with respect to the amplitudes in a time width in which the spectrum has a spectrum shape with a peak in the first respiration and a spectrum shape without a peak in the second respiration, and
estimating a respiratory state of the user based on the spectrum.
